(19) United States
(12) Patent Application Publication
(10) Pub. No.: US 2024/0416108 A1
(43) Pub. Date: Dec. 19, 2024

(54) BLOOD PUMPS FOR USE WITH PEDIATRIC PATIENTS

(71) Applicant: ABIOMED, Inc., Danvers, MA (US)

(72) Inventors: Sonya Sanat Bhavsar, Danvers, MA (US); Scott Corbett, Danvers, MA (US); Caitlyn Hastie, Danvers, MA (US); Christopher W. Sheils, Danvers, MA (US); Vincent Roy, Danvers, MA (US)

(73) Assignee: ABIOMED, Inc., Danvers, MA (US)

(21) Appl. No.: 18/742,384

(22) Filed: Jun. 13, 2024

Related U.S. Application Data
(60) Provisional application No. 63/521,011, filed on Jun. 14, 2023.

(51) Int. Cl. A61M 60/857; A61M 60/117; A61M 60/174; A61M 60/237; A61M 60/515; A61M 60/531; A61M 60/829

(52) U.S. Cl. CPC A61M 60/857; A61M 60/117; A61M 60/174; A61M 60/237; A61M 60/515; A61M 60/531; A61M 60/829

(57) ABSTRACT

Various devices, systems, and methods are disclosed. The method may include determining a body surface area of a patient (who may have a body surface area of about 0.6 m² to about 1.3 m²), selecting a blood pump from a plurality of differently sized blood pumps based on the body surface area of the patient, implanting the blood pump, and activating the blood pump to provide support to the patient. The selected blood pump may have a cannula connecting an inlet and an outlet. The cannula may have a bend and a cannula length measuring an axial length between the inlet and the outlet. The cannula length may be between about 30 mm to about 46 mm if the body surface area is less than 0.9 m².

### CROSS-REFERENCE TO RELATED APPLICATIONS

The present application claims priority to 63/521,011, filed Jun. 14, 2023, the contents of which are incorporated by reference herein in its entirety.

### TECHNICAL FIELD

Embodiments disclosed herein relate to medical devices, such as intravascular blood pumps.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the Impella® heart pump or blood pump. Some blood pump assemblies may be introduced percutaneously through the vascular system during a cardiac procedure. Specifically, blood pump assemblies can be inserted via a catheterization procedure through, e.g., the femoral artery, the axillary/subclavian artery, etc., into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY

In various aspects, a method may be provided. The method may include determining a body surface area of a patient. The method may include selecting a blood pump from a plurality of differently sized blood pumps based on the body surface area of the patient. The blood pump may have an inlet, and an outlet opposite the inlet. The outlet may be connected to the inlet by a cannula. The cannula may have a bend and a cannula length measuring an axial length between the inlet and the outlet.

Selecting the blood pump may include selecting a blood pump having a cannula length of between about 70 millimeters and about 78 millimeters if the body surface area is greater than about 0.8 square meters. Selecting a blood pump may include selecting a blood pump where the cannula length is 6.0 centimeters if the body surface area is greater than about 1.1 square meters. Selecting a blood pump may include selecting a blood pump where the cannula length is between about 30 millimeters to about 46 millimeters if the body surface area is less than 0.9 square meters. Selecting a blood pump may include selecting a blood pump where the cannula length is between about 34 millimeters to about 42 millimeters if the body surface area is less than 0.9 square meters. Selecting a blood pump may include selecting a blood pump where the cannula length is between about 36 millimeters and 39 millimeters if the body surface area is less than 0.9 square meters.

The method may include implanting the blood pump. Implanting the blood pump may include implanting the blood pump via a midclavicular axillary line.

The method may include activating the blood pump to provide support to the patient.

The blood pump may have an atraumatic distal end. The atraumatic distal end may include a pigtail coupled to the inlet. The pigtail may have a pigtail length that is measured from the inlet to a distalmost point on the pigtail. The pigtail length may be between about 10 millimeters and about 18 millimeters if the body surface area is less than 0.9 square meters. The pigtail length may be between about 12 millimeters and about 16 millimeters if the body surface area is less than 0.9 square meters. The pigtail length may be between about 13 millimeters and 15 millimeters if the body surface area is less than 0.9 square meters.

The bend in the cannula may be between about 125 degrees and about 145 degrees if the body surface area is less than 0.9 square meters. The bend in the cannula is between about 130 degrees and about 140 degrees if the body surface area is less than 0.9 square meters. The bend in the cannula may be between about 134 degrees and about 136 degrees if the body surface area is less than 0.9 square meters.

In various aspects, a system may be provided. The system may include a blood pump. The blood pump may include an inlet, and an outlet positioned opposite the inlet. The blood pump may include a cannula connecting the inlet and the outlet. The cannula may have a length between the inlet and the outlet. The blood pump may be configured to be inserted into a patient having a body surface area of between about 0.6 square meters and about 1.3 square meters. The blood pump may be implanted via a midclavicular axillary line. The body surface area may be between about 0.7 square meters and about 1.2 square meters. The body surface area may be between about 0.86 square meters and 1.15 square meters.

The system may include a pigtail coupled to the inlet. The pigtail may have a pigtail length measured by a distance between the inlet and a distalmost point on the pigtail.

In various aspects, a blood pump may be provided. The blood pump may include an inlet, and an outlet opposite the inlet. The blood pump may include a cannula connecting the inlet and the outlet. The cannula may include a first region, a second region. There may be a cannula angle (i.e., a bend of less than 180 degrees) between the first region and the second region. The cannula may have a cannula length measured by summing the total axial lengths of the first region and the second region. The blood pump may include an atraumatic distal tip. The atraumatic distal tip may include a pigtail coupled to the inlet. The pigtail may have a pigtail length measured by a distance between the inlet and a distalmost point on the pigtail. The cannula length may be between about 30 millimeters to about 46 millimeters, and the pigtail length may be between about 10 millimeters to about 18 millimeters.

The cannula length may be about 34 millimeters to about 42 millimeters, and the pigtail length may be between about 12 millimeters and about 16 millimeters. The cannula length may be between about 36 millimeters and about 39 millimeters and the pigtail length may be between about 13 millimeters and about 15 millimeters.

The cannula angle may be between about 125 degrees and about 145 degrees. The cannula angle may be between about 130 degrees and about 140 degrees. The cannula angle may be between about 134 degrees and about 136 degrees.

The blood pump may be configured for use in patients with a body surface area of less than 0.9 square meters. The blood pump may be configured to be implanted via a midclavicular axillary line.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1A shows an illustrative circulatory support device that may be used in accordance with some embodiments.

FIG. 1B illustrates the circulatory support device of FIG. 1A positioned within a heart of a patient.

FIG. 1C illustrates a ventricular support system including the circulatory support device of FIG. 1A.

FIG. 2 illustrates transvalvular fit and delivery for a patient receiving mechanical circulatory support.

FIG. 3 illustrates a method according to some embodiments.

### DETAILED DESCRIPTION

As is known, pediatric patients with advanced heart failure face a high risk of death and morbidity. As also known, there are few mechanical circulatory support options available. For example, not all existing mechanical circulatory support devices may fit a pediatric patient's anatomy. Specifically, some existing mechanical circulatory support devices may be configured for larger patients (e.g., adolescent and/or adult patients) and may be too large for a pediatric patient's vasculature and/or ventricular anatomy.

Accordingly, the inventors have recognized the benefit of a device that is sized to accommodate smaller patients (e.g., pediatric patients). For example, the inventors have recognized the benefit of a blood pump with geometry and/or proportions that are configured to accommodate the small anatomy of pediatrics. The inventors have also recognized the benefit of a higher blood flow through the cannula because of a shorter cannula. The inventors have also recognized the benefit of determining the appropriate support device for each patient. For example, in some embodiments, a patient's body surface area (BSA), such as a pediatric patient's BSA, may be calculated in order to determine if the patient's anatomy is suitable for one of a plurality of differently sized support devices. In this regard, a physician can appropriately fit patient to an adult or adolescent-sized blood pump or to a pediatric-sized blood pump.

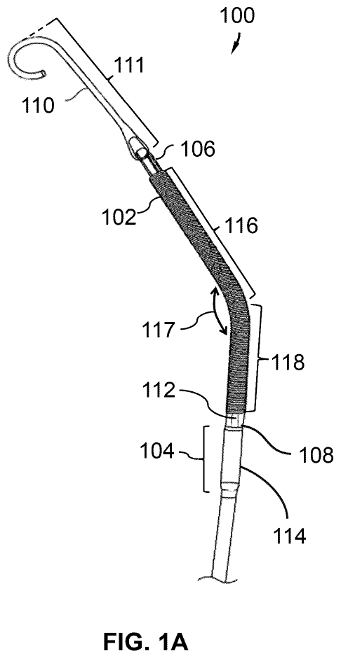

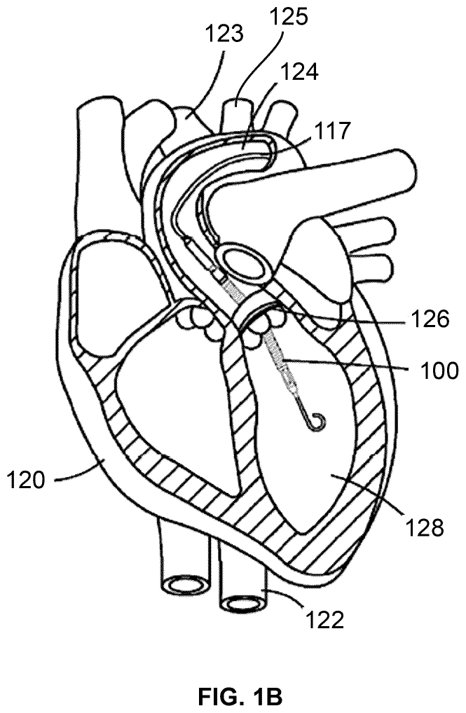

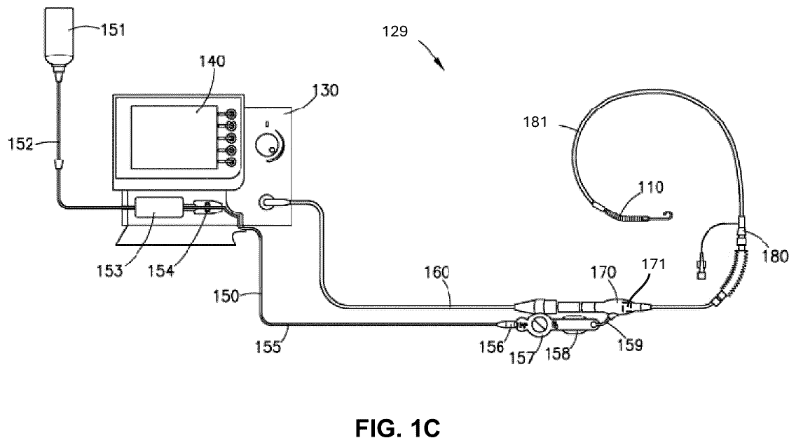

Turning to the figures, FIG. 1A shows a view of a blood pump (100) of a blood pump system according to some embodiments. The blood pump (100) may include a cannula (102), a pump housing (104), a blood flow inlet (106), a blood flow outlet (108), and an atraumatic tip, such as a pigtail (110). The cannula (102) may be connected to the blood flow inlet (106) on the distal portion and the blood flow outlet (108) at the proximal portion of the cannula (102). The pump housing (104) may include an impeller (112) which is disposed inside of the blood flow outlet (108).

In some embodiments, as shown in this view, the pump housing (104) may include an on-board motor (114). In other embodiments, the blood pump (100) may include an extracorporeal motor.

In some embodiments, the blood pump (100) may not include a pigtail (e.g., the blood pump may be free of a pigtail). In some embodiments, the blood pump may be free of an atraumatic tip.

As shown in FIG. 1B, the blood pump (100) may be positioned in a patient's heart (120). For example, blood pump (100) may be inserted percutaneously via the femoral artery (122) into the ascending aorta (124), across the aortic valve (126), and into the left ventricle (128). In other implementations, a blood pump may, for example, be inserted percutaneously via the axillary artery (123) into the ascending aorta (124), across the aortic valve (126), and into the left ventricle (128). In other implementations, the blood pump may be inserted via a carotid artery (such as common carotid artery (125) or a carotid artery connected to the common carotid artery). In other implementations, a blood pump may, for example, be inserted directly into the ascending aorta (124), across the aortic valve (126), and into the left ventricle (128).

During operation, blood pump (100) may entrain blood from the left ventricle (128) and expel blood into the ascending aorta (124). As a result, blood pump (100) may perform some of the work normally done by the patient's heart (120). The hemodynamic effects of blood pumps may include an increase in cardiac output, improvement in coronary blood flow resulting in a decrease in left ventricle end-diastolic pressure, pulmonary capillary wedge pressure, myocardial workload, and oxygen consumption.

As shown in FIG. 1C, the blood pump (100) may form part of a ventricular support system (129). Ventricular support system (129) also may include a controller (130) (e.g., an AUTOMATED IMPELLA CONTROLLER®, referred to herein as “AIC,” from ABIOMED, Inc., Danvers, Mass.), a display (140), a purge subsystem (150), a connector cable (160), a plug (170), and a repositioning unit (180). As shown, controller (130) may include display (140). Controller (130) may be configured to monitor and control operation of blood pump (100). During operation, purge subsystem (150) may be configured to deliver a purge fluid to blood pump (100) through a catheter tube to prevent blood from entering the motor (not shown) within motor housing. In some implementations, the purge fluid is a dextrose solution (e.g., 5% dextrose in water with 25 or 50 IU/mL of heparin). Connector cable (160) may provide an electrical connection between blood pump (100) and controller (130).

Plug (170) may connect catheter tube (181), purge subsystem (150), and connector cable (160). In some implementations, plug (170) may include a storage device (171) (e.g., a memory) configured to store, for example, operating parameters to facilitate transfer of the patient to another controller if needed. Repositioning unit (180) may be used to reposition blood pump (100) in the patient's heart.

As shown, in some embodiments, the ventricular support system may include a purge subsystem (150) having a container (151), a supply line (152), a purge cassette (153), a purge disc (154), purge tubing (155), a check valve (156), a pressure reservoir (157), an infusion filter (158), and a sidearm (159). Container (151) may, for example, be a bag or a bottle. As will be appreciated, in other embodiments the ventricular support system may not include a purge subsystem. In some embodiments, a purge fluid may be stored in container (151). Supply line (152) may provide a fluidic connection between container (151) and purge cassette (153).

Purge cassette (153) may control how the purge fluid in container (151) is delivered to blood pump (100). For example, purge cassette (153) may include one or more valves for controlling a pressure and/or flow rate of the purge fluid. Purge disc (154) may include one or more pressure and/or flow sensors for measuring a pressure and/or flow rate of the purge fluid.

As shown, controller (130) may include purge cassette (153) and purge disc (154). Purge tubing (155) may provide a fluidic connection between purge disc (154) and check valve (156). Pressure reservoir (157) may provide additional filling volume during a purge fluid change. In some implementations, pressure reservoir (157) may include a flexible rubber diaphragm that provides the additional filling volume by means of an expansion chamber. Infusion filter (158) may help prevent bacterial contamination and air from entering catheter tube (181). Sidearm (159) provides a fluidic connection between infusion filter (158) and plug (170).

Although shown as having separate purge tubing and connector cable, it will be appreciated that in some embodiments, the ventricular support system may include a single connector with both fluidic and electric lines connectable to the AIC.

As illustrated in FIG. 1A, the cannula (102) may have a first cannula region (116) and a second cannula region (118). In some embodiments, the cannula (102) may further include a bend angle (117). The bend angle (117) may be the angle between the longitudinal axis of the first cannula region (116) and the longitudinal axis of the second cannula region (118). The cannula (102) may have a cannula length that is equal to the sum of the axial lengths of the first cannula region (116) and the second cannula region (118). As will be appreciated, while two regions are used here, any number of suitable regions may be used. In such embodiments, the cannula length may be equal to the sum of the axial length of each of the regions of the cannula, regardless of the number of regions.

Furthermore, the pigtail (110) may have a pigtail length (111). The pigtail length (111) may be equal to the distance from the distal end of the blood flow inlet (106) to the distalmost point of the pigtail (110).

In some embodiments, the cannula length and the pigtail length (111) may be varied to result in different sized blood pumps for various pediatric patients. The blood pump having the appropriate dimensions may be selected based on BSA.

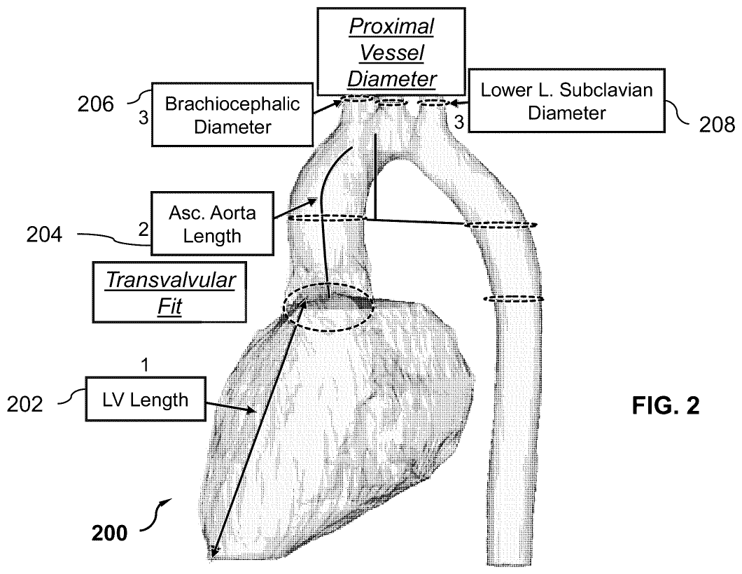

A patient's heart (200) is illustrated in FIG. 2. The patient's BSA may be correlated with the patient's left ventricle length (202), ascending aorta length (204), brachiocephalic diameter (206), and left subclavian ostial diameter (208). For example, in some embodiments, a cannula length of 74.3±0.2 millimeters and a pigtail length of about 40 millimeters may fit the anatomy of a patient having a BSA greater than 0.86 square meters. As will be appreciated, any suitable intravascular blood pump having a cannula length of 74.3±0.2 millimeters may be used, for example, an Impella CP. In such embodiment, a patient having a BSA greater than 0.86 square meters may have a left ventricle length (202) that may accommodate the cannula length and the pigtail length. In other embodiments, a blood pump having no pigtail and a cannula length of 86 millimeters may fit the anatomy of a patient having a BSA of 1.15 square meters. As will be appreciated, any suitable intravascular blood pump having a cannula length of 86 millimeters may be used, for example, an Impella 5.5. In such embodiment, a patient having a BSA of 1.15 square meters may have a left ventricle length (202) that may not be able to accommodate a larger cannula and a pigtail and thus a blood pump having no pigtail and a shorter cannula may be used. In some embodiments, the patient may have a BSA of less than 0.9 square meters and thus a pump having different dimensions and/or geometry than commercially available intravascular blood pumps may be required.

In patients having a BSA less than 0.9 square meters, the blood pump may have a different cannula length, pigtail length (111), and/or bend angle (117). In one embodiment, the cannula length may be between about 30 millimeters and about 46 millimeters. As a result of a shortened cannula length, higher blood flow through the cannula is achieved. In some embodiments, the pigtail length may be between about 10 millimeters and about 18 millimeters. In some embodiments, the bend angle may be between about 125 degrees and about 145 degrees. As will be appreciated, the cannula length, pigtail length, and/or bend angle may be any suitable value. As will also be appreciated, other dimensions and/or geometry may be modified in addition to and/or instead of the aforementioned dimensions.

In some embodiments, the blood pump may be introduced or implanted via femoral access or any other suitable method. In some embodiments, the blood pump may be introduced or implanted via axillary artery. In some embodiments, the blood pump may be introduced or implanted via carotid artery. In some embodiments, the blood pump may be introduced or implanted directly (e.g., directly into or through the ascending aorta, etc.). In other embodiments, the blood pump may be implanted surgically via a graft sutured to the aortic valve. In some embodiments, the patient may have a BSA that is below 1.15 meters, with the patient's brachiocephalic diameter (206) and/or the left subclavian ostial diameter (208) being too small to accommodate certain blood pumps. In such embodiments, the blood pump may be implanted via midclavicular axially access. In other embodiments, the blood pump may be implanted via carotid access.

As will be appreciated in view of the above, in some embodiments, the first and second lengths and the bend angle may be configured to support various patient populations. In that regard, the shape and size of the blood pump, or portions of the blood pump may be varied depending upon the size of the patient. Accordingly, in some embodiments, a method of determining patient eligibility and fit for intravascular blood pump support is described.

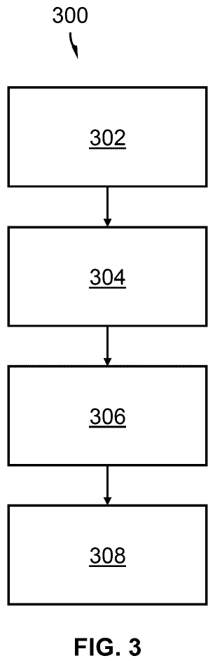

An exemplary method is illustrated in FIG. 3, which may include a method (300) of using a blood pump on a patient necessitating hemodynamic support, such as a pediatric patient. In some embodiments, the method (300) may include determining (302) a body surface area of the patient.

As will be appreciated, the body surface area of the patient may be determined using any suitable method. For example, in Du Bois, D., et al., “A formula to estimate the approximate surface area if height and weight be known” (Published 1 Jun. 1916), Medicine, JAMA Internal Medicine, a formula for body surface area based on weight and height of a person was estimated based on the following formula: BSA (m²)=0.007184× weight^{0.425}×height^{0.725}, where weight is in kg and height is in cm. Other similar approaches (e.g., estimating BSA utilizing height and weight of a person) include, e.g., Mosteller's formula: BSA=square root of ((height× weight)/3600), Haycock's formula: BSA=0.024265×weight^{0.5378}×height^{0.3964}, Gehan and George's formula: BSA=0.0235×weight^{0.51456}×height^{0.42246}, or Fujimoto's formula: BSA=0.008883×weight^{0.444}×height^{0.663}. Other approaches are envisioned. For example, in some embodiments, the BSA estimates are based solely on body weight. See, e.g., El Edelbi, R. A., et al., “Estimation of body surface area in neonates, infants, and children using body weight alone”, Int J Pediatr Adolesc Med. 2021 December; 8 (4): 221-228, where three options are discussed, including a third order polynomial equation (BSA=B0+B1*weight+B2*weight²+B3*weight³), a Meeh type equation (BSA=A×weight^{B}), and a Boyd self-adjusting type equation (BSA=A×weight^{BClog(weight)}.

The blood pump is configured to be inserted into a patient having a body surface area of between about 0.6 square meters and about 1.3 square meters. In some embodiments, the body surface area may be between about 0.7 square meters and about 1.2 square meters. In some embodiments, the body surface area may be between about 0.86 square meters and 1.15 square meters.

In some embodiments, the method may also include selecting (304) an appropriately sized blood pump based on the body surface area of the patient. In such embodiments, the blood pump may be similar to those described herein and may have an inlet and an outlet that may be opposite the inlet. The outlet may be connected to the inlet by a cannula having a bend and a length that is measured by the distance between the bend and the inlet. In some embodiments, the method may also include implanting (306) the blood pump. In such embodiments, the blood pump may be implanted via the midclavicular axillary line. However, as will be appreciated, the blood pump may be implanted via any suitable means. In some embodiments, the method may include activating (308) the blood pump to provide support to the patient.

In some embodiments, selecting (304) the blood pump may include selecting a blood pump having a prescribed length that is chosen based upon the body surface area of the patient.

In some embodiments, the length of the blood pump may be one of two lengths (i.e., a relatively shorter length if the body surface area is below a predetermined first threshold, and a relatively longer length if the body surface area is above a predetermined second threshold. In some embodiments, the total cannula length of the blood pump may be one of three lengths. For example, in one embodiment, the blood pump may have a cannula length of 6.0 centimeters if the body surface area is greater than 1.1 square meters. In another embodiment, the blood pump may have a cannula length between about 4.6 centimeters and 6.0 centimeters if the body surface area is 0.9 square meters to 1.1 square meters. In another embodiment, the blood pump may have a cannula length between about 3.0 centimeters and about 4.6 centimeters if the body surface area is less than 0.9 square meters. In such embodiment, the blood pump may have a cannula length that is 3.8 centimeters if the body surface area is less than 0.9 square meters.

In some embodiments, the blood pump may include a pigtail that may be coupled to the inlet. In such embodiments, the pigtail may have a pigtail length that is equal to distance between the inlet and the distal end of the pigtail. In some embodiments, the pigtail length may be between about 1.0 centimeters and about 1.8 centimeters if the body surface area is less than 0.9 square meters. In such embodiments, the pigtail length may be 1.4 centimeters if the body surface area is less than 0.9 square meters.

In some embodiments, the blood pump may have a cannula length of between about 70 millimeters and about 78 millimeters if the body surface area is greater than about 0.8 square meters.

In some embodiments, the blood pump may have a cannula length of about 30 millimeters to about 46 millimeters if the body surface area is less than 0.9 square meters. In some embodiments, the blood pump may have a cannula length of about 34 millimeters to about 42 millimeters if the body surface area is less than 0.9 square meters. In some embodiments, the blood pump may have a cannula length between about 36 millimeters and 39 millimeters if the body surface area is less than 0.9 square meters.

In some embodiments, the pigtail length (measured from the inlet to a distalmost point on the pigtail) may be between about 10 millimeters and about 18 millimeters if the body surface area is less than 0.9 square meters. In some embodiments, the pigtail length may be between about 12 millimeters and about 16 millimeters if the body surface area is less than 0.9 square meters. In some embodiments, the pigtail length may be between about 13 millimeters and 15 millimeters if the body surface area is less than 0.9 square meters.

In some embodiments, the cannula may have a bend. In such embodiments, the bend may be between about 125 degrees and about 145 degrees if the body surface area is less than 0.8 square meters. In such embodiments, the cannula bend may be 135 degrees. In some embodiments, the bend in the cannula may be between about 125 degrees and about 145 degrees if the body surface area is less than 0.9 square meters. In some embodiments, the bend in the cannula may be between about 130 degrees and about 140 degrees if the body surface area is less than 0.9 square meters. In some embodiments, the bend in the cannula may be between about 134 degrees and about 136 degrees if the body surface area is less than 0.9 square meters.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method, comprising:
determining a body surface area of a patient;
selecting a blood pump from a plurality of differently sized blood pumps based on the body surface area of the patient, the blood pump having an inlet and an outlet opposite the inlet, the outlet connected to the inlet by a cannula, the cannula having a bend and a cannula length measuring an axial length between the inlet and the outlet;
implanting the blood pump; and
activating the blood pump to provide support to the patient.

2. The method of claim 1, wherein implanting the blood pump further comprises implanting the blood pump via a midclavicular axillary line.

3. The method of claim 1, wherein selecting a blood pump includes selecting a blood pump having a cannula length of between about 70 millimeters and about 78 millimeters if the body surface area is greater than about 0.8 square meters.

4. The method of claim 1, wherein selecting a blood pump includes selecting a blood pump where the cannula length is 6.0 centimeters if the body surface area is greater than about 1.1 square meters.

5. The method of claim 1, wherein selecting a blood pump includes selecting a blood pump where the cannula length is between about 30 millimeters to about 46 millimeters if the body surface area is less than 0.9 square meters.

6. The method of claim 5, wherein selecting a blood pump includes selecting a blood pump where the cannula length is between about 34 millimeters to about 42 millimeters if the body surface area is less than 0.9 square meters.

7. The method of claim 6, wherein selecting a blood pump includes selecting a blood pump where the cannula length is between about 36 millimeters and 39 millimeters if the body surface area is less than 0.9 square meters.

8. The method of claim 1, wherein the blood pump has a pigtail coupled to the inlet, the pigtail having a pigtail length that is measured from the inlet to a distalmost point on the pigtail.

9. The method of claim 8, wherein the pigtail length is between about 10 millimeters and about 18 millimeters if the body surface area is less than 0.9 square meters.

10. The method of claim 9, wherein the pigtail length is between about 12 millimeters and about 16 millimeters if the body surface area is less than 0.9 square meters.

11. The method of claim 10, wherein the pigtail length is between about 13 millimeters and 15 millimeters if the body surface area is less than 0.9 square meters.

12. The method of claim 1, wherein the bend in the cannula is between about 125 degrees and about 145 degrees if the body surface area is less than 0.9 square meters.

13. The method of claim 12, wherein the bend in the cannula is between about 130 degrees and about 140 degrees if the body surface area is less than 0.9 square meters.

14. The method of claim 13, wherein the bend in the cannula is between about 134 degrees and about 136 degrees if the body surface area is less than 0.9 square meters.

15. A system comprising:
a blood pump comprising:
an inlet and an outlet positioned opposite the inlet; and
a cannula connecting the inlet and the outlet, the cannula having a length between the inlet and the outlet;
wherein the blood pump is configured to be inserted into a patient having a body surface area of between about 0.6 square meters and about 1.3 square meters.

16. The system of claim 15, wherein the blood pump is implanted via a midclavicular axillary line.

17. The system of claim 15, wherein the body surface area is between about 0.7 square meters and about 1.2 square meters.

18. The system of claim 17, wherein the body surface area is between about 0.86 square meters and 1.15 square meters.

19. The system of claim 15, further comprising a pigtail coupled to the inlet, the pigtail having a pigtail length measured by a distance between the inlet and a distalmost point on the pigtail.

20. A blood pump, comprising:
an inlet and an outlet opposite the inlet;
a cannula connecting the inlet and the outlet, the cannula further comprising a first region, a second region, and a cannula angle between the first region and the second region, the cannula having a cannula length measured by a total axial length of the first region and the second region; and
a pigtail coupled to the inlet, the pigtail having a pigtail length measured by a distance between the inlet and a distalmost point of the pigtail;
wherein the cannula length is between about 30 millimeters to about 46 millimeters and the pigtail length is between about 10 millimeters to about 18 millimeters.

21-27. (canceled)
